(19) United States
(12) Patent Application Publication
(10) Pub. No.: US 2023/0355228 A1
(43) Pub. Date: Nov. 9, 2023

(54) SUTURE-TO-BUTTON ASSEMBLIES FOR PERFORMING SURGICAL PROCEDURES

(71) Applicant: ARTHREX, INC., Naples, FL (US)

(72) Inventor: Michaela LAMBCKE, Naples, FL (US)

(21) Appl. No.: 17/735,336

(22) Filed: May 3, 2022

(51) Int. Cl. A61B 17/04

(52) U.S. Cl. CPC A61B 17/0401; A61B 2017/0404; A61B 2017/0464; A61B 2017/0495

(57) ABSTRACT

Suture-to-button assemblies are disclosed for performing various surgical procedures. For example, suture-to-button assemblies may be utilized for performing tissue (e.g., tendon, ligament, cartilage, etc.) repairs. Exemplary suture-to-button assemblies may include a suture tail adapted for fixating a suture construct to a button of the assembly. The suture-to-button assemblies may be used alone or as part of a tensionable surgical system during the surgical procedures.

### BACKGROUND

This disclosure relates generally to the field of surgery, and more particularly to suture-to-button assemblies for performing surgical procedures.

Surgical intervention may be required when tissue such as ligament or tendon detaches from bone. Techniques that have been developed for performing tissue repairs generally involve tying the tissue back to bone using suture and one or more fixation devices (e.g., anchors, buttons, bone plates, etc.).

### SUMMARY

This disclosure relates to suture-to-button assemblies that may be used for performing surgical procedures, such as tissue repairs, for example.

An exemplary suture-to-button assembly for performing surgical procedures may include, inter alia, a button, and a suture construct fixated to the button. The suture construct includes a primary suture strand and a filler suture strand spliced through the primary suture strand to establish a tail portion adapted for fixating the suture construct to the button.

An exemplary method for fixating a suture to a button may include, inter alia, threading a first suture strand through a first hole of a button, splicing a second suture strand through the first suture strand to form a suture construct that is received within the first hole, and cutting a portion of the suture construct, thereby forming a tail portion that extends outside of the first hole of the button. The tail portion is adapted for knotlessly fixating the suture construct to the button.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an exemplary suture-to-button assembly for performing surgical procedures.

FIG. 2 is a top view of a button of a suture-to-button assembly.

FIG. 3 is a first end view of the button of FIG. 2.

FIG. 4 is a second end view of the button of FIG. 2.

FIG. 5 illustrates a first step of a method for fixating a suture construct to a button to form a suture-to-button assembly.

FIG. 6 illustrates another step of the method for fixating the suture construct to the button.

FIG. 7 illustrates another step of the method for fixating the suture construct to the button.

FIG. 8 illustrates yet another step of the method for fixating the suture construct to the button.

FIG. 9 illustrates an exemplary tensionable surgical system that includes a suture-to-button assembly.

### DETAILED DESCRIPTION

This disclosure is directed to suture-to-button assemblies for performing various surgical procedures. For example, the exemplary suture-to-button assemblies described herein may be utilized for performing tissue (e.g., tendon, ligament, cartilage, etc.) repairs. An exemplary suture-to-button assembly may include a suture tail adapted for fixating a suture construct to a button of the suture-to-button assembly. The suture-to-button assemblies may be used either alone or as part of a tensionable surgical system during surgical procedures. These and other features of this disclosure are described in further detail below.

An exemplary suture-to-button assembly for performing surgical procedures may include, inter alia, a button and a suture construct fixated to the button. The suture construct includes a primary suture strand and a filler suture strand spliced through the primary suture strand to establish a tail portion adapted for fixating the suture construct to the button.

In a further embodiment, the tail portion establishes a thickened portion of the suture construct.

In a further embodiment, the tail portion includes a first outer diameter, and a first hole of the button through which the suture construct extends includes a second outer diameter. The first outer diameter is larger than the second outer diameter.

In a further embodiment, the button includes a first end wall having a first hole and a second end wall having a second hole.

In a further embodiment, the suture construct is accommodated within the first hole.

In a further embodiment, the first hole is connected to a third hole of the button, and the third hole opens through a top surface and a bottom surface of the button.

In a further embodiment, the second hole is configured to receive a tip portion of an inserter of the suture-to-button assembly.

In a further embodiment, the tail portion is stiffened by an adhesive.

In a further embodiment, the primary suture strand includes a suture tape, and the filler suture strand includes a round suture.

In a further embodiment, the filler suture strand is accommodated within a lumen of an outer jacket of the primary suture strand.

In a further embodiment, the suture-to-button assembly is part of a tensionable surgical system that further includes a second button and a suture loop construct. The suture loop construct connects to the button and the second button.

In a further embodiment, the suture construct is spliced through the suture loop construct.

An exemplary method for fixating a suture to a button may include, inter alia, threading a first suture strand through a first hole of a button, splicing a second suture strand through the first suture strand to form a suture construct that is received within the first hole, and cutting a portion of the suture construct, thereby forming a tail portion that extends outside of the first hole of the button. The tail portion is adapted for knotlessly fixating the suture construct to the button.

In a further embodiment, threading the first suture strand includes positioning the first suture strand within an additional hole of the button prior to passing the first suture strand through the first hole.

In a further embodiment, the first hole is connected to the additional hole.

In a further embodiment, the method includes applying an adhesive to an outer jacket of the first suture strand prior to cutting the portion of the suture construct.

In a further embodiment, cutting the suture construct includes cutting along a cut line to form the tail portion, wherein the cut line is located about 0.5 mm to about 5 mm outside of the first hole.

In a further embodiment, the method includes tensioning a free strand end of the second suture strand to seat the second suture strand within the tail portion.

In a further embodiment, upon completing the splicing, the second suture strand extends beyond a length of the first suture strand.

In a further embodiment, the method includes splicing the suture construct through a suture loop construct to form a tensionable surgical system.

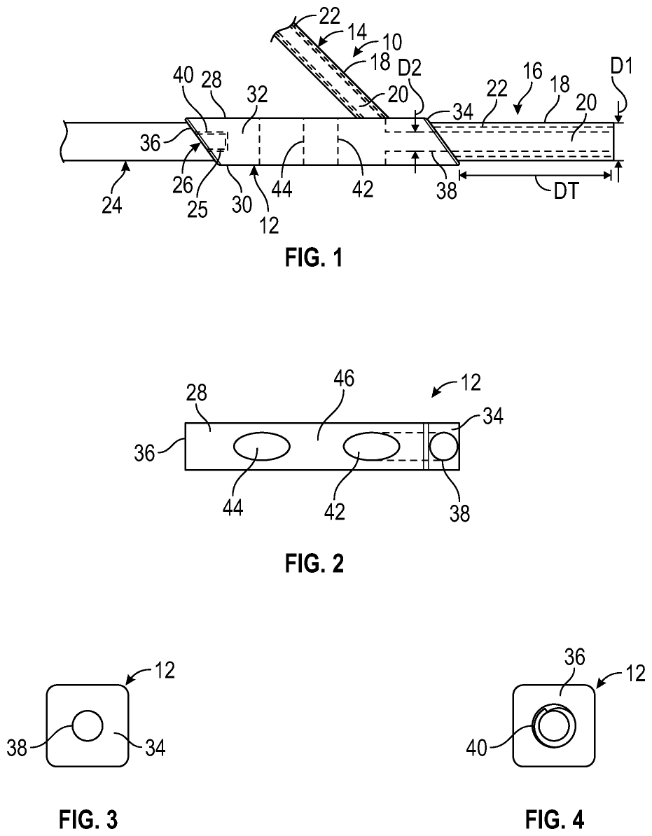

FIG. 1 illustrates an exemplary suture-to-button assembly 10 that may be utilized when performing various surgical procedures. The suture-to-button assembly 10 may be used to perform tissue repairs, such as when ligament or tendon becomes detached from bone or when cartilage tears occur, for example. Other types of surgical procedures are contemplated within the scope of this disclosure. The suture-to-button assemblies described herein could be utilized to perform any surgical procedure associated with any joint of the human musculoskeletal system.

The suture-to-button assembly 10 may include a button 12 and a suture construct 14 fixated to the button 12. The suture construct 14 may be passed through one or more openings formed in the button 12 and may include a tail portion 16 that extends outside of the button 12. As further detailed herein, the tail portion 16 may be configured to fixedly secure the suture construct 14 to the button 12. The suture construct 14 is therefore knotlessly secured to the button 12.

The suture construct 14 may include multiple strands of high strength suture. In an embodiment, the suture construct 14 includes a primary suture strand 18 and a filler suture strand 20. The filler suture strand 20 may be spliced through a portion of the primary suture strand 18 and is thus accommodated within a lumen 22 of the primary suture strand 18 in order to form the tail portion 16.

The primary suture strand 18 may be a braided suture tape, such as FiberTape®. The filler suture strand 20 may be a round suture, such as FiberWire®. FiberTape® and FiberWire® are suture products marketed and sold by Arthrex, Inc. However, other types of sutured or other flexible strands could be utilized as part of the suture construct 14 within the scope of this disclosure.

Splicing the filler suture strand 20 through the primary suture strand 18 may thicken portions of the suture construct 14 as compared to non-spliced portions of the primary suture strand 18. For example, the tail portion 16 may establish a thickened portion of the suture construct 14. The tail portion 16 may therefore function as a “hard stop” for substantially preventing the suture construct 14 from separating from the button 12. Despite being “thickened,” the tail portion 16 provides a relatively compact profile that is less bulky and more flexible than other types of suture fixation constructs, such as knots, for example.

The tail portion 16 may extend outside of the button 12 by a distance DT. In an embodiment, the distance DT is between about 0.5 mm and about 5 mm. In another embodiment, the distance DT is between about 2 mm and about 5 mm. In another embodiment, the distance DT is about 2 mm. In yet another embodiment, the distance DT is about 5 mm. The actual value of the distance DT could vary depending on various design specific parameters, including but not limited to the desired fixation strength of the suture construct 14 relative to the button 12. In this disclosure, the term “about” means that the expressed quantities or ranges need not be exact but may be approximated and/or larger or smaller, reflecting acceptable tolerances, conversion factors, measurement error, etc.

The suture-to-button assembly 10 may further include an inserter 24. The inserter 24 may be utilized for inserting the suture-to-button assembly 10 within a pilot hole formed in a bone during tissue repairs or other surgical procedures, for example. In an embodiment, a tip portion 26 of the inserter 24 is configured to engage a portion of the button 12 that is located on an opposite side of the button 12 from the tail portion 16. However, other configurations are also contemplated within the scope of this disclosure.

An exemplary design of the button 12 of the suture-to-button assembly 10 is illustrated in FIGS. 1, 2, 3, and 4. Although an exemplary design is shown and described herein, the button 12 could include any size, shape, and configuration within the scope of this disclosure. Therefore, the specific size, shape, and overall configuration of the button 12 shown in FIGS. 1-4 are not intended to limit this disclosure.

The button 12 may include an oblong-shaped body having a top surface 28, a bottom surface 30, a side wall 32 extending between the top surface 28 and the bottom surface 30, a first end wall 34, and a second end wall 36. The top surface 28 and the bottom surface 30 may be substantially flat surfaces of the button 12, and the first end wall 34 and the second end wall 36 may be slanted or angled relative to the top surface 28 and the bottom surface 30.

A first hole 38 may be formed in the first end wall 34, and a second hole 40 may be formed in the second end wall 36. The first hole 38 may be a round, non-threaded opening of the button 12 for accommodating the suture construct 14, and the second hole 40 may be a round, threaded opening of the button 12 for accommodating the inserter 24.

A third hole 42 and a fourth hole 44 may be formed completely through the body of the button 12. The third and fourth holes 42, 44 therefore open through both the top surface 28 and the bottom surface 30 of the button 12. The third hole 42 and the fourth hole 44 may be separated by a bridge 46 of the button 12. The third hole 42 and the fourth hole 44 may be elongated, non-threaded openings of the button 12.

The first hole 38 may open into the third hole 42 within an interior section of the button 12. The first hole 38 and the third hole 42 are thus considered to be connected to one another.

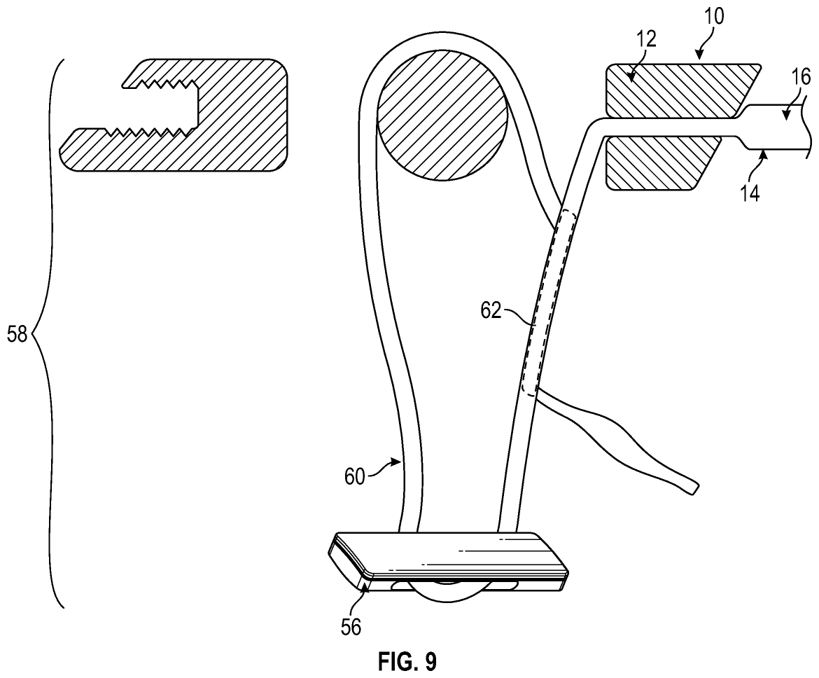

Referring now primarily to FIG. 1, the suture construct 14 may be received at least partially within the third hole 42 and completely through the first hole 38 of the button 12. However, other configurations are contemplated, and a worker of ordinary skill in the art would understand that any of the openings of the button 12 could be configured to accommodate portions of the suture construct 14. Moreover, although not shown in FIG. 1, an additional suture construct, such as a suture loop, could be accommodated within the third and fourth holes 42, 44 of the button 12 (see, for example, FIG. 9).

The tail portion 16 of the suture construct 14 may include an outer diameter D1, and the first hole 38 of the button 12 may include an outer diameter D2. The outer diameter D1 may be larger than the outer diameter D2 for retaining the suture construct 14 to the button 12. In another embodiment, the tail portion 16 may be larger than the outer diameter of the button 12.

The tip portion 26 of the inserter 24 may engage the second hole 40 of the button 12. In an embodiment, the tip portion 26 may include an engagement mechanism 25 (e.g., a threaded shaft) for detachably engaging an inner threading of the second hole 40.

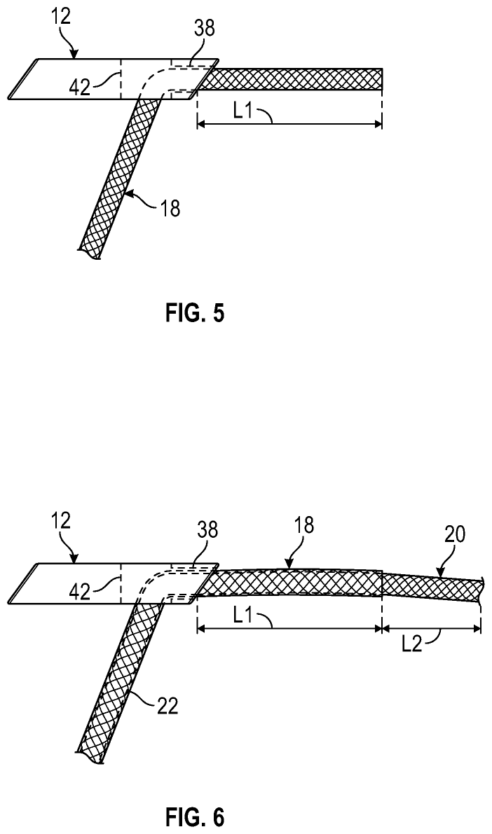

FIGS. 5, 6, 7, and 8, with continued reference to FIGS. 1-4, illustrate an exemplary method for fixating the suture construct 14 to the button 12 to form the suture-to-button assembly 10. A greater or fewer number of assembly steps than described below may be utilized, and the exact order of steps shown in not intended to limit this disclosure.

Referring first to FIG. 5, the primary suture strand 18 may be passed through the button 12. In an embodiment, the primary suture strand 18 is positioned within the third hole 42 and is then passed from the third hole 42 through the first hole 38. A length L1 of the primary suture strand 18 therefore extends outside of the button 12 from the first hole 38.

Next, as shown in FIG. 6, the filler suture strand 20 may be spliced through a portion of the primary suture strand 18. The filler suture strand 20 may be accommodated within the lumen 22 of the primary suture strand 18 and therefore also passes through the first hole 38 of the button 12. A length L2 of the filler suture strand 20 may extend beyond the length L1 of the primary suture strand 18 once the splicing is complete. Needles or other splicing devices may be used to splice the filler suture strand 20 through the primary suture strand 18.

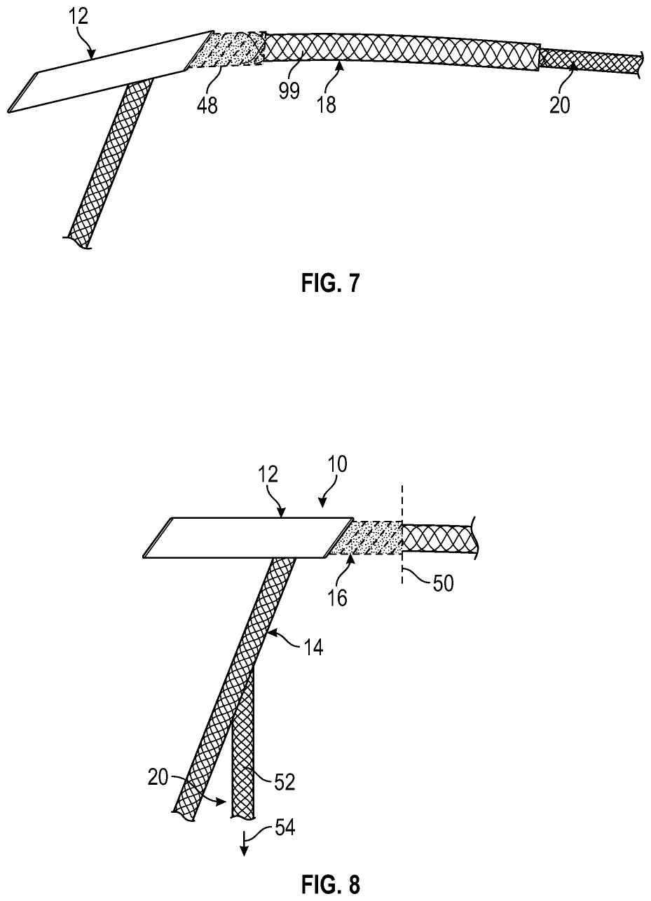

Referring now to FIG. 7, an adhesive 48 may next be applied to an outer jacket 99 of the primary suture strand 18 along a portion thereof that protrudes outside of the first hole 38. Given the braided nature of the outer jacket 99, the adhesive 48 may penetrate into the lumen 22 and thus adhere to the portions of the filler suture strand 20 that are spliced through the primary suture strand 18. The adhesive 48 thus helps secure the filler suture strand 20 in place relative to the primary suture strand 18 and creates a more rigid effect. In an embodiment, the adhesive 48 is LOCTITE® 4014. However, other types of adhesives could alternatively be utilized within the scope of this disclosure.

Referring now to FIG. 8, the suture construct 14 may be cut along a cut line 50 to form the tail portion 16. The tail portion 16 may be slightly stiffened by virtue of the adhesive 48. The location of the cut line 50 may be selected in order for the tail portion 16 to extend outside of the button 12 by the desired distance DT. A free strand end 52 of the filler suture strand 20 may then be tensioned in a direction of arrow 54, thereby further seating the filler suture strand 20 within the tail portion 16 due to applied internal tensile forces. Upon completion of this step, the suture-to-button assembly 10 is considered fully assembled and may be used for performing surgical procedures.

The suture-to-button assembly 10 described above may be utilized in combination with an additional fixation device (e.g., a second button) to create a tensionable surgical system for performing some types of surgical procedures. An exemplary tensionable surgical system 58 is illustrated in FIG. 9 and may include the suture-to-button assembly 10, a second button 56, and a suture loop construct 60. The suture loop construct 60 may be secured to and extend between the button 12 of the suture-to-button assembly 10 and the second button 56. The suture construct 14 of the suture-to-button assembly 10 may be spliced through the suture loop construct 60 at a spliced section 62 to establish the tensionable surgical system 58.

The suture-to-button assemblies described herein may be utilized to perform a variety of surgical procedures. The proposed assemblies provide improved designs for fixating suture to buttons or other fixation devices. The designs provide a more compact and flexible design compared to known suture fixation mechanisms while providing adequate fixation strength.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. A suture-to-button assembly for performing surgical procedures, comprising:
a button; and
a suture construct fixated to the button,
wherein the suture construct includes a primary suture strand and a filler suture strand spliced through the primary suture strand to establish a tail portion adapted for fixating the suture construct to the button.

2. The suture-to-button assembly as recited in claim 1, wherein the tail portion establishes a thickened portion of the suture construct.

3. The suture-to-button assembly as recited in claim 1, wherein the tail portion includes a first outer diameter, and a first hole of the button through which the suture construct extends includes a second outer diameter, and further wherein the first outer diameter is larger than the second outer diameter.

4. The suture-to-button assembly as recited in claim 1, wherein the button includes a first end wall having a first hole and a second end wall having a second hole.

5. The suture-to-button assembly as recited in claim 4, wherein the suture construct is accommodated within the first hole.

6. The suture-to-button assembly as recited in claim 5, wherein the first hole is connected to a third hole of the button, and further wherein the third hole opens through a top surface and a bottom surface of the button.

7. The suture-to-button assembly as recited in claim 4, wherein the second hole is configured to receive a tip portion of an inserter of the suture-to-button assembly.

8. The suture-to-button assembly as recited in claim 1, wherein the tail portion is stiffened by an adhesive.

9. The suture-to-button assembly as recited in claim 1, wherein the primary suture strand includes a suture tape and the filler suture strand includes a round suture.

10. The suture-to-button assembly as recited in claim 1, wherein the filler suture strand is accommodated within a lumen of an outer jacket of the primary suture strand.

11. A tensionable surgical system, comprising:
the suture-to-button assembly of claim 1;
a second button; and
a suture loop construct connected to the button and the second button.

12. The tensionable surgical system as recited in claim 11, wherein the suture construct of the suture-to-button assembly is spliced through the suture loop construct.

13. A method for fixating a suture to a button, comprising:
threading a first suture strand through a first hole of a button;
splicing a second suture strand through the first suture strand to form a suture construct that is received within the first hole; and
cutting a portion of the suture construct, thereby forming a tail portion that extends outside of the first hole of the button,
wherein the tail portion knotlessly fixates the suture construct to the button.

14. The method as recited in claim 13, wherein threading the first suture strand includes:
positioning the first suture strand within an additional hole of the button prior to passing the first suture strand through the first hole.

15. The method as recited in claim 14, wherein the first hole is connected to the additional hole.

16. The method as recited in claim 13, comprising:
applying an adhesive to an outer jacket of the first suture strand prior to cutting the portion of the suture construct.

17. The method as recited in claim 13, wherein cutting the portion of the suture construct includes:
cutting the suture construct along a cut line to form the tail portion, wherein the cut line is about 0.5 mm to about 5 mm outside of the first hole.

18. The method as recited in claim 17, comprising:
tensioning a free strand end of the second suture strand to seat the second suture strand within the tail portion.

19. The method as recited in claim 13, wherein, upon completing the splicing, the second suture strand extends beyond a length of the first suture strand.

20. The method as recited in claim 13, comprising:
splicing the suture construct through a suture loop construct to form a tensionable surgical system.
